(19) United States
(12) Patent Application Publication
(10) Pub. No.: US 2006/0210509 A1
(43) Pub. Date: Sep. 21, 2006

(54) Hair conditioner

(76) Inventors: Andress K. Johnson, Pleasantville, NY (US); Phuong-Nga Trinh, Newtonville, MA (US); Bruce Beard, Hopewell Junction, NY (US)

Correspondence Address: RALPH J. MANCINI;AKZO NOBEL INC., 7 LIVINGSTONE AVENUE, DOBBS FERRY, NY, 10522-3408, US

(21) Appl. No.: 11/084,967

(22) Filed: Mar. 21, 2005

(51) Int. Cl. A61K 8/89; A61K 8/41

(52) U.S. Cl. 424/70.12; 424/70.28

(57) ABSTRACT

The present invention generally relates to a silicone-based hair conditioning composition having improved silicone hair conditioner deposition upon the hair. The composition of the invention comprises hair conditioning compositions having dispersed, non-volatile silicone conditioning agents, cationic conditioning agents and at least one nonionic amphiphilic polymer having at least one fatty chain and at least one hydrophilic unit.

### FIELD OF THE INVENTION

The present invention is related to hair conditioning compositions having dispersed, non-volatile silicone conditioning agents, at least one cationic conditioning agent and at least one nonionic amphiphilic polymer having at least one fatty chain and at least one hydrophilic unit.

### BACKGROUND OF THE INVENTION

Human hair is constantly exposed to damaging processes both physical and chemical. Physical processes that damage the hair include heat drying with a hair dryer or hot iron, brushing and combing. While chemical processes include shampooing, permanent weaving, dyeing and bleaching. All of these processes result in morphological damage to the hair fibers reducing the natural suppleness and manageability.

Shampooing hair cleans by removing excess soil and sebum. However, the shampooing process has disadvantages. Shampooing can cause hair to be in a tangled and unmanageable state, especially when the hair is damaged. Shampooing can also cause hair dryness due to the removal of natural oils or other hair moisturizing materials. After shampooing, the hair can also suffer from a perceived loss of “softness”. Softness, of course, is a generally desirable attribute for many users of shampoo products.

A variety of approaches have been developed to alleviate the after-shampoo problems. These range from the inclusion of hair conditioning aids in shampoos to post-shampoo application of hair conditioners, i.e., hair rinses. Hair rinses typically work by depositing a polymeric film, cationic hair conditioning surfactant, or other material onto the hair fiber.

Stable, silicone-containing hair conditioning compositions have been reasonably successful in the marketplace in that they can provide excellent hair conditioning benefits to the user. However, it is desirable to improve these types of conditioners by increasing the efficiency of the silicone hair conditioning component in order to reduce the amount of silicone that is incorporated into the shampoo thereby reducing raw materials cost. The most important factor affecting effectiveness of the silicone hair conditioner is the ability of the silicone to deposit upon the hair fiber.

It is therefore an object of this invention to provide a more efficient deposition of silicone on the hair fiber.

### SUMMARY OF THE INVENTION

The present invention generally relates to a silicone-based hair conditioning composition having improved silicone hair conditioner deposition upon the hair. The composition of the invention comprises hair conditioning compositions having dispersed, non-volatile silicone conditioning agents, cationic conditioning agents and at least one nonionic amphiphilic polymer having at least one fatty chain and at least one hydrophilic unit.

### DETAILED DESCRIPTION OF THE INVENTION

The compositions of the present invention comprise dispersed insoluble, nonvolatile silicone hair conditioning agent, at least one nonionic amphiphilic polymer having at least one fatty chain and at least one hydrophilic unit and water. The silicone conditioning agent is typically suspended in the composition with a suspending aid. Surprisingly, the combination of a cationic surfactant and a nonionic amphiphilic polymer having at least one fatty chain and at least one hydrophilic unit improves the deposition of the silicone hair conditioning agent upon the hair.

The conditioning composition of the present invention typically contains from about 0.01% to about 10% of a nonvolatile, insoluble silicone hair conditioning agent; from 0.1 to 15% of one or more organic, cationic surfactants useful in conditioning hair, preferably 0.5 to 8%, more preferably 1-5%; and from about 0.01 to 10% by weight relative to the total weight of the conditioning composition of at least one nonionic amphiphilic polymer having at least one fatty chain and at least one hydrophilic unit; more preferably, 0.2 to 5% by weight; the remainder being water.

Silicone Conditioning Agent

The silicone conditioning agent employable in the context of the present invention is preferably a nonvolatile, insoluble silicone conditioning agent. The conditioner compositions, in particular, will generally comprise from about 0.01% to about 10%, by weight, of the silicone conditioning agent, in another embodiment from about 0.05% to about 5%, in another embodiment from about 0.05% to about 3%, and in still another embodiment from about 0.1% to about 2.5% of the silicone conditioning agent. The silicone conditioning agent comprises a nonvolatile, insoluble silicone fluid. The silicone conditioning agent for use herein in conditioning compositions will generally have an average viscosity of from about 10 to 100,000 centistokes at 25° C., in another embodiment from about 200 to about 60,000 centistokes, and in still another embodiment from about 500 to about 5,000 centistokes.

As used hereinafter, the term “insoluble” in reference to the silicone conditioning agent shall mean that the silicone material is not soluble in water. The term “nonvolatile” in reference to the silicone conditioning agent as used herein shall be interpreted according to the meaning well understood to those skilled in the art, i.e., the silicone fluid exhibits very low or no significant vapor pressure at ambient conditions. The term “silicone fluid” shall mean flowable silicone materials having a viscosity of less than 100,000 centistokes at 25° C. The term “silicone”, as used herein, shall be synonomous with the term “polysiloxane”.

Suitable nonvolatile silicone fluids for use in hair conditioning agents include polyalkyl siloxanes, polyaryl siloxanes, polyalkylaryl siloxanes, polyether siloxane copolymer and mixtures thereof. However, other silicone fluids having hair conditioning properties may be used. The nonvolatile polyalkyl siloxane fluids that may be used include, but are not limited to polydimethylsiloxanes. These siloxanes are available from the General Electric Company as a Viscasil series and from Dow Corning as the Dow Corning 200 series.

The polyalkylaryl siloxane fluids employable in the context of the invention include, but are not limited to, polymethylphenylsiloxanes. These siloxanes are available from the General Electric Company as SF 1075 methyl phenyl fluid or from Dow Corning as 556 Cosmetic Grade Fluid.

The polyether siloxane copolymer include, a polypropylene oxide modified dimethylpolysiloxane (e.g., Dow Corning DC-1248) although ethylene oxide or mixtures of ethylene oxide and propylene oxide may also be used. The ethylene oxide and polypropylene oxide level must be sufficiently low to prevent solubility in water and the composition hereof.

Silicone fluids hereof also include polyalkyl or polyaryl siloxanes having the following structure: 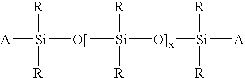
wherein R is alkyl or aryl, and x is an integer from about 7 to about 8,000. “A” represents groups which block the ends of the silicone chains.

The alkyl or aryl groups substituted on the siloxane chain (R) or at the ends of the siloxane chains (A) may have any structure as long as the resulting silicones remain fluid at room temperature, are hydrophobic, are neither irritating, toxic nor otherwise harmful when applied to the hair, are compatible with the other components of the composition, are chemically stable under normal use and storage conditions, and are capable of being deposited on and of conditioning hair.

Suitable A groups include methyl, methoxy, ethoxy, propoxy, and aryloxy. The two R groups on the silicone atom may represent the same group or different groups. Preferably, the two R groups represent the same group. Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred.

Cationic silicone fluids may be used, although nonionic silicone fluids are preferred.

Cationic Conditioning Surfactant

The compositions of the present invention also comprise one or more organic, cationic surfactants useful for the conditioning of hair, hereinafter “cationic conditioning agent”. The cationic conditioning agents of the invention include, but are not limited to those selected quaternary ammonium surfactants and amino surfactants that are positively charged at the pH of the conditioning composition. The compositions will generally contain from about 0.1% to about 15%, preferably from about 0.5% to about 8%, more preferably from about 1% to about 5%, of the soluble cationic conditioning agent.

The preferred cationic conditioning surfactants for use in the present invention are those which are useful for providing conditioning benefits, particularly hair conditioning properties and which are quaternary ammonium or amino compounds having at least one N-radical.

One preferred class of cationic compounds is commonly referred to as quaternary esterquats. Various types of ester containing quaternary ammonium compounds can be employed in the context of the present invention including triester quaternary ammonium compounds (TEQ) and diester quaternary ammonium compounds (DEQ). Quaternary ammonium compounds having particularly good performance and stability profiles are obtained by reaction of C₁₂-C₂₂ fatty acids or the hydrogenation products thereof, or a mixture of such acids, with an alkanolamine in the presence of an acid catalyst, wherein the ratio of fatty acid to alkanolamine is from about 1.40-2.1. The resultant esteramine reaction products are subsequently quaternized to obtain the quaternary ammonium salts of the present invention. Esterquats of this type can be found in U.S. Pat. No. 6,770,608 which is incorporated herein by reference.

TEQ quaternary ammonium salts employable in the invention generally comprise a mixture mono-(I), di-(II) and tri-ester (III) components of the following formulae: 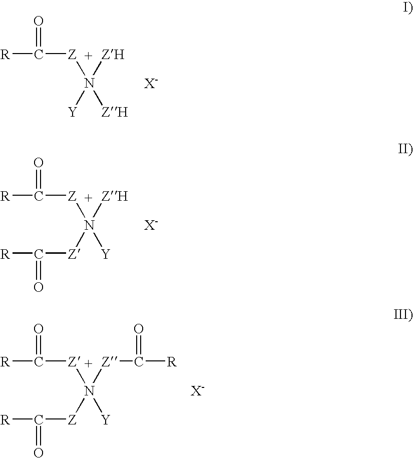
wherein:
Z, Z′ and Z″ are the same or different and are selected from straight or branched chain, optionally substituted oxyalkylene or polyoxyalkylene groups having from 2-6 carbon atoms, preferably 3-6 carbon atoms, where the oxyalkylene units number from about 1-10, preferably 1-5, and still more preferably 1-2; each R group is individually selected from straight or branched chain, saturated or unsaturated, optionally substituted alkyl groups having from 11 to 23 carbon atoms, Y is an alkylphenyl group or a straight or branched chain optionally substituted C₁ to C₆ alkyl or alkylene group; and X⁻ represents a softener compatible anion including but not limited to halogen, CH₃SO₄ or C₂H₅SO₄.

Other ester-containing quaternary ammonium compounds employable in the context of the present invention include, but are not limited to Diester Quaternary Ammonium Compounds (DEQ) of the formula 1 or 2:
[R′₄₋ₘ—N⁽⁺⁾—[(CH₂)ₙ—Y—R″]ₘX⁽⁻⁾ 1.
where each R′ is selected from the group consisting of C₁ to C₆ alkyl or hydroxyalkyl groups; each m is 2 or 3; each n is 1 to 4; each R″ is selected from the group consisting of C₁₂ to C₂₂ hydrocarbyl or substituted hydrocarbyl groups; each Y is —O—(O)C— or —C(O)—O and X⁻ is a compatable anion, or 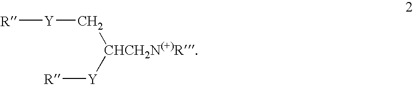
wherein each of Y, R′, R″ and X— have the meanings defined above and where R′″ is a hydrocarbon group containing 11 to 31 carbon atoms.

Additionally, Diesterdiquats of the following formula 3 can also be usefully employed in the context of the present invention: 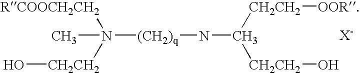
where R″ and X⁻ have the meanings defined above and q is an integer of from 2-6.

Salts of primary, secondary and tertiary fatty amines also comprise a preferred cationic surfactant material. The alkyl groups of such amines preferably have from about 1 to about 30 carbon atoms and must contain at least one, preferably 2 to about 10, nonionic hydrophilic moieties selected from alkoxy, polyoxyalkylene, alkylamido, hydroxyalkyl, and alkylester moieties, and mixtures thereof. Secondary and tertiary amines are preferred, tertiary amines are particularly preferred. Specific examples of suitable amines include diethyl aminoethyl polyoxyethylene (5) laurate, coco-polyglyceryl-4 hydroxypropyl dihydroxy ethylamine, and dihydroxyethyl tallowamine hydrochloride.

For shampoo formulations it is preferred that quaternary ammonium or amino compounds having at least one N-radical further contain one or more nonionic hydrophilic moieties selected from alkoxy, polyoxyalkylene, alkylamido, hydroxyalkyl, and alkylester moieties, and combinations thereof. The surfactant contains at least one hydrophilic moiety within 4 (inclusive), preferably within 3 (inclusive), carbon atoms of the quaternary nitrogen or cationic amino nitrogen. For purposes herein, this means that the closest non-carbon atom in the hydrophilic moiety to the cationic nitrogen must be within the stated number of carbon atoms relative to said nitrogen. Additionally, carbon atoms that are part of a hydrophilic moiety, e.g., carbon atoms in a hydrophilic polyoxyalkylene (e.g., —CH₂—CH₂—O—), that are adjacent to other hydrophilic moieties are not counted as when determining the number of hydrophilic moieties within 4, or preferably 3, carbon atoms of the cationic nitrogen. In general, the alkyl portion of any hydrophilic moiety is preferably a C₁-C₃ alkyl. Suitable hydrophile-containing radicals include, for example, ethoxy, propoxy, polyoxyethylene, polyoxypropylene, ethylamido, propylamido, hydroxymethyl, hydroxyethyl, hydroxypropyl, methylester, ethylester, propylester, or mixtures thereof, as nonionic hydrophile moieties.

Additional classes of quaternary ammonium cationic surfactants useful herein are those of the general formula: 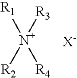
wherein R₁, R₂, R₃ and R₄ radicals comprise, independently, substituted or unsubstituted alkylchains of from 1 to about 30 carbon atoms, or a alkyl having from 1 to about 30 carbon atoms and containing one or more aromatic, ether, ester, amido, or amino moieties present as substituents or as linkages in the radical chain, wherein at least one of the R₁—R₄ radicals contains one or more hydrophilic moieties selected from alkoxy (preferably C₁-C₃ alkoxy), polyoxyalkylene (preferably C₁-C₃ polyoxyalkylene), alkylamido, alkylamidopropyl, hydroxyalkyl, alkylester, and combinations thereof. Optionally R₄ can be hydrogen. X is a soluble salt forming anion preferably selected from halogen (especially chlorine), acetate, citrate, phosphate, nitrate, sulfonate, and alkyl sulfate radicals.

Another class of useful quaternary ammonium salt surfactants include those of the formula: 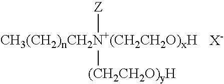
wherein n is from 8-28, preferably 16, x+y=2 to about 15. Z is a short chain alkyl, preferably a C₁-C₃ alkyl, more preferably methyl, and X is a water soluble salt forming anion (e.g., Cl, sulfate, etc.) Z can be optionally hydrogen.

Another class of quaternary ammonium salt surfactants include those of the Formula: 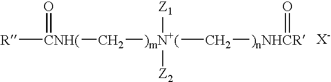
wherein Z₁ and Z₂ are, independently, substituted or unsubstituted hydrocarbyls, and, preferably, Z₁ is an alkyl, preferably a C₁-C₃ alkyl, more preferably methyl, and Z₂ is a short chain hydroxyalkyl, preferably hydroxymethyl or hydroxyethyl, n and m independently are integers from 2 to 4, inclusive, preferably from 2 to 3, inclusive, more preferably 2, R′ and R″, independently, are substituted or unsubstituted hydrocarbyls, preferably C₁₂-C₂₀ alkyl or alkenyl, and X is a soluble salt-forming anion (e.g., sulfate, Cl, etc.). Z₁ or Z₂ can optionally be hydrogen.

Still other class of quaternary ammonium salt surfactants are of the formulas: 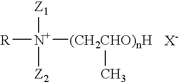
wherein R is a hydrocarbyl, preferably a C₁-C₃ alkyl, more preferably ethyl, Z₁ and Z₂ are, independently, short chain hydrocarbyls, preferably C₂-C₄ alkyl or alkenyl, more preferably ethyl, n is from about 2 to about 40, preferably from about 7 to about 30, and X is a soluble salt-forming anion, as set forth previously; 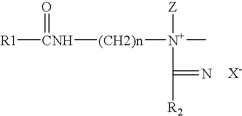
wherein R₁ and R₂, independently, are C₁₂-C₂₀ hydrocarbyls, preferably C₁₆-C₁₈ alkyl or alkenyls (e.g., those derived from tallow acid), Z is a C₁-C₃ hydrocarbyl, preferably ethyl, n is 2 or 3, and X is a soluble salt forming anion; and 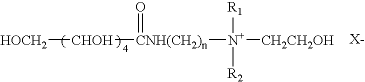
wherein n is 2 or 3, R₁ and R₂, independently are C₁-C₃ hydrocarbyls preferably ethyl, and X is as defined above.

Specific examples of useful quaternary ammonium salts include polyoxyethylene (2) stearyl ethyl ammonium chloride, methyl bis(hydrogenated tallowamidoethyl) 2-hydroxyethyl ammonium methyl sulfate, polyoxypropylene (9) diethyl methyl ammonium chloride, tripolyoxyethylene (total PEG=10) stearyl ammonium phosphate, bis(N-hydroxyethyl-2-oleyl imidazolinium chloride) polyethylene glycol (12), and isododecylbenzyl triethanolammonium chloride.

Other ammonium quaternary and amino surfactants include those of the above general formula 1 in the form of ring structures formed by covalently linking of the radicals. Examples of such cationic surfactants include imidazolines, imidazoliniums, and pyridiniums, etc., wherein said surfactant has at least one nonionic hydrophile-containing radical as set forth above. Specific examples include 2-heptadecyl-4,5-dihydro-1H-imidazol-1-ethanol, 4,5-dihydro-1-(2-hydroxyethyl)-2-isoheptadecyl-1-phenylmethylimidazolium chloride, and 1-[2-oxo-2-[[2-[(1-oxooctadecyl)oxy]ethyl]aminoethyl] pyridinium chloride.

Generally, the pH of the conditioner compositions will be less than about 10, typically from about 2 to about 9, preferably from about 3.5 to about 5.5. The cationic surfactant, of course, must remain cationic upon application to the hair in order for there to be adequate substantivity between the conditioning agent and the hair. The cationic conditioning agents for use herein may also include a plurality of ammonium quaternary moieties or amino moieties, or a mixture thereof.

Nonionic Amphiphilic Polymer

The nonionic amphiphilic polymers containing at least one fatty chain and at least one hydrophilic unit employable in the invention are well known in the art. Suitable examples include, but are not limited to:
(1) hydroxypropyl guars modified with groups containing at least one fatty chain, such as the product Esaflor HM 22 (C₂₂ alkyl chain) sold by the company Lamberti, and the products Miracare XC95-3 (C14 alkyl chain) and RE205-1 (C₂₀ alkyl chain) sold by the company Rhone-Poulenc.
(2) polyether urethanes containing at least one fatty chain such as C₈-C₃₀ alkyl or alkenyl groups, for instance the products Dapral T210 and Dapral T212, now known respectively as Elfacos T210 and Elfacos T212, sold by the company Akzo Nobel. Other examples would be Bermocoll PUR series, sold by Akzo Nobel.
(3) copolymers of vinylpyrrolidone and of hydrophobic monomers containing a fatty chain; non-limiting examples include: Antaron V216 or Ganex V216 (vinylpyrrolidone/hexadecene copolymer) sold by the company ISP; Antaron V220 or Ganex V220 (vinylpyrrolidone/eicosene copolymer) sold by the company ISP.
(4) copolymers of C₁-C₆ alkyl methacrylates or acrylates and of amphiphilic monomers containing at least one fatty chain, such as, for example, the oxyethylenated methyl methacrylate/stearyl acrylate copolymer sold by the company Goldschmidt under the name Antil 208.
(5) copolymers of hydrophilic methacrylates or acrylates and of hydrophobic monomers containing at least one fatty chain, such as, for example, the polyethylene glycol methacrylate/lauryl methacrylate copolymer.
The nonionic amphiphilic polymers containing at least one fatty chain and at least one hydrophilic unit according to the invention are preferably used in an amount which may range approximately from 0.01 to 10% by weight relative to the total weight of the conditioning composition. More preferably, this amount varies approximately from 0.2 to 5% by weight.

Polyether urethanes containing at least one fatty chain such as C₈-C₃₀ alkyl or alkenyl groups, for instance the products Dapral T210 and Dapral T212, now known respectively as Elfacos T210 and Elfacos T212, sold by the company Akzo Nobel, are a particularly useful class of nonionic amphiphilic polymers.

Emulsifier and Surfactant

Emulsifier/surfactants are those that provide various functions such as improving the stability, texture/consistency as well as other aesthetic properties. Examples of emulsifier/surfactants include anionic surfactants such as sodium cetearyl sulfate and the like, and alkyl phosphates as well as alkyl ether phosphates; amphoterics and betaines such as cocoamphoglycinate, cocoamidopropyl betaine and the like; and nonionics such as fatty alcohols, ethoxylated and/or propoxylated fatty alcohols and fatty esters, alkylamdiopropylamines and the like. A suitable amount of emulsifier/surfactant is required to obtain a stable hair conditioning formulation. A minimum of 4.5% of emulsifier/surfactant is preferred to formulate a stable formulation, wherein a portion of said emulsifier/surfactant can come from and/or be provided by the cationic surfactant.

Water

Water is generally present at a level of from about 20% to about 95%, preferably from about 50% to about 85%, more preferably from about 60% to about 85%, by weight of the composition.

Optional Components

The compositions herein can contain a variety of non-essential optional components. Such optional ingredients include, for example, preservatives such as benzyl alcohol, methyl paraben, propyl paraben and imidazolidinyl urea; cationic conditioning agents, including cationic conditioning polymers; fatty alcohols; block polymers of ethylene oxide and propylene oxide such as Pluronic F88 offered by BASF Wyandotte; sodium chloride, sodium sulfate; ammonium zylene sulfonate; propylene glycol; polyvinyl alcohol; ethyl alcohol; Polyquaternium-10 (an industry term designated by The Cosmetic, Toiletry and Fragrance Association (CTFA) for the polymeric quaternary ammonium salt of hydroxyethyl cellulose reacted with trimethyl ammonium substituted epoxide), commercially available from Union Carbide Corp. (Danbury, Conn., USA) under their UCARE POLYMER JR series of materials, e.g., UCARE POLYMER JR-30M, JR-125 and JR-400; pH adjusting agents such as citric acid, succinic acid, phosphoric acid, sodium hydroxide, sodium carbonate, etc.; perfumes; dyes; and sequestering agents such as disodium ethylenediamine tetraacetate. These optional ingredients are typically used at levels of from about 0.01% to about 10% of the composition. This list of optional ingredients is not meant to be exclusive, and other optional components can be utilized.

The pH of the present compositions is not generally critical and may be in the range of from 2 to about 10, preferably from about 2 to about 9, more preferably from about 3.5 to about 5.5.

Method of Use

The present compositions are used in a conventional manner for conditioning hair. An effective amount of the composition for conditioning hair, typically, from about 1 g to about 20 g of the composition, is applied to hair that has preferably cleaned with a shampoo and then rinsed out. Application to the hair typically includes working the composition through the hair such that most or all of the hair is contacted with the composition then rinse with water.

### EXAMPLE 1

The present inventors have identified a synergy between quaternary ammonium salts and a nonionic polymer (alkoxylated urethane Eflacos T212) that improved conditioning of silicone based formulations. Panel tests conducted with the following formulation showed preference for the formulation with the alkoxylated urethane over a National brand (Tresseme) and the formulation without polymer.

Test Formulation

| | |
|---|---|
| Citric acid | 0.14% |
| Stearylamidopropyl dimethylamine | 0.8% |
| Stearyl alcohol | 1.9% |
| Cetyl alcohol | 0.8% |
| Glycerl stearate | 0.7% |
| Steareth-20 | 0.3% |
| Di-palmitoylethyldimonium chloride | 2.75% |
| PPG-14 Palmeth-60 hexyl dicarbamate | 0.6% |
| Dimethicone | 0.9% |
| Water | qs |

The formulation is prepared by heating the water to 65° C. then adding the citric acid and stearamidopropyl dimethylamine. Once completely dispersed then heated to 70° C. and combine the cetyl alcohol, stearyl alocohol, glyceryl stearate and stearch-20. Then cool to 60° C. and add the Di-palmitoylethyldimonium chloride, PPG-14 Palmeth-60 hexyl dicarbamate and dimethicone. Cool to 40° C. and add optional ingredients such as perfume, preservatives and dyes.

Methodology

Medium brown European hair tresses were obtained from International Hair Importers & Products Inc, New York. The hair tresses were bleach damaged with a generic formulation containing hydrogen peroxide, SLS, bisulfide and ammonia. The application of the conditioner shown above was done by the following methods:
Dispersion—damaged hair tresses were exposed to a 10% dispersion of the conditioner formulation with and without polymer for 60 seconds at 35-40° C. then rinsed for 30 seconds at 35-40° C.
Direct—1 gram of conditioner formulation with and without polymer was directly rubbed onto the damaged hair tresses for 60 seconds then rinsed for 30 seconds at 35-40° C.

Combing Tests

Panel tests—Hair tresses that had been exposed to a 10% dispersion of the hair conditioner with and without polymer or national brand were placed side by side into clamps, then using a comb with wide teeth were combed to remove all the knots. A set of 10-14 panelists compared the tresses on ease of combing using the narrow teeth on the comb and indicated a preference.

Wet Comb—The wet combing force was measured by using the load cell of a Dia-Stron Miniature Tensile Tester (MTT). When the comb is pulled through a wet hair tress, reduction in total work done is compared to an untreated hair tress.

Dry Comb—The dry combing force was measured by using the load cell of a Dia-Stron Miniature Tensile Tester (MTT). When the comb is pulled through a dry hair tress, reduction in total work done is compared to an untreated hair tress.

XPS Analysis

XPS analysis was performed on mounted hair fibers from various treated hair tresses using a Physical Electronic 5600 spectrometer. Low resolution elemental composition scans were collected first to identify the elements present at the surface of the fiber. Subsequent high resolution scans were performed to obtain chemical state information for the identification of specific chemical structures of the hair or conditioner molecules on the hair fiber.

SEM

Electron microscope images were collected with a Zeiss EVO-50 XVP instrument. Images for this work were collected with a secondary electron detector. Probe beam energy was 20 KV. A thin coating of Au was evaporated onto the surface of the fibers to minimize charge buildup. Magnifications of 10K× of the samples treated using both application methods with and without the polymer.

Results and Discussion

Initial panel tests using dispersion application of the hair condition found a 66% preference for the hair conditioner with the polymer against a national brand. A repeat study found a 64% preference of the formulation containing the polymer over the formulation without. XPS analysis found higher silicone levels on the hair tresses exposed to the dispersed conditioner formulation with the polymer compared to the formula without polymer. This data is consistent with the results of the panel test preference study. The formulation containing the polymer does not show any quaternary nitrogen or characteristic hair fiber sulfur/nitrogen signals, indicating a uniform silicone over layer coverage of greater than 7 nm on the hair fiber. The formulation without the polymer shows both the quaternary nitrogen and characteristic hair fiber sulfur/nitrogen signals indicating a patchy and/or thinner layer of the silicone with an estimated thickness of 4 nm.

### XPS Results: Deposition of Silicone and Quaternary Nitrogen on Hair Fibers

| | Silicone (Atom %) | | Quaternary Nitrogen (Atom %) | |
|---|---|---|---|---|
| Formula | Dispersion | Direct | Dispersion | Direct |
| With polymer | 19.8 | 3.35 | 0 | 0.97 |
| Without polymer | 18.9 | 1.85 | 0.4 | 0.77 |

The results demonstrate the application method significantly influences the deposition of the silicone and quaternary ammonium salts onto the hair fiber. The direct application shows significantly lower levels of silicone on the hair fiber. However, the conditioner formulation with the polymer still has twice the deposited silicone compared to the formulation without polymer. The amount of quaternary nitrogen appears higher in the hair fibers treated with the conditioner with the polymer, but additional data indicates this may not be significant.

In order to confirm the benefits of the nonionic polymer in direct application a combing experiment was conducted. The results of the wet comb show no significant difference between the conditioner with polymer and without the polymer. The dry comb does show a 10% reduction in the tresses exposed to the conditioner with the polymer.

### Influence of Nonionic Polymer on Combing Force Reduction

Combing Force Reduction (%)

| Formula | Wet | Dry |
|---|---|---|
| With polymer | 77 | 53 |
| Without polymer | 79 | 42 |

Hair fibers treated with the conditioner with and without the polymer were viewed under the electron microscope as shown below:

[image]

The hair fiber exposed to the conditioner with the polymer shows a smoother cuticle surface and a cementing of the cuticle that is not seen in the conditioner without the polymer. These morphological effects are not influenced by the application method, but their magnitude and extent on the surface is.

What is claimed is:

1. A hair conditioning composition, comprising:
(a) from about 0.1% to about 15%, by weight of at least one cationic conditioning agent;
(b) from about 0.01% to about 10%, by weight, of at least one nonvolatile, insoluble, silicone conditioning agent having a viscosity, at 25° C., of from about 10 centipoise to about 100,000 centipoise;
(c) from about 0.01 to about 10% of at least one nonionic amphiphilic polymers containing at least one fatty chain and at least one hydrophilic unit; and
(d) the remainder water.

2. The composition of claim 1, further comprising an emulsifier.

3. The composition of claim 2, wherein said emulsifier is an anionic surfactant, a nonionic surfactant, an amphoteric surfactant, a betaine, or a mixture thereof.

4. The composition of claim 3 wherein the emulsifier is sodium cetearyl sulfate, alkyl phosphate, alkyl ether phosphates, cocoamphoglycinate, cocoamidopropyl betaine, fatty alcohol, ethoxylated fatty alcohol, propoxylated fatty alcohol, fatty ester, alkylamidopropylamine, or a mixture thereof.

5. The composition of claim 3 wherein said composition comprises a minimum of 4.5% of emulsifier.

6. The composition of claim 1 wherein the silicone conditioning agent is a polyalkyl siloxane, polyaryl siloxane, polyalkylaryl siloxane, polyether siloxane copolymer, polydimethylsiloxanes, polymethylphenylsiloxanes, polypropylene oxide modified dimethylpolysiloxane, or a mixture thereof.

7. The composition of claim 1 wherein the silicone conditioning agent is of the following structure: 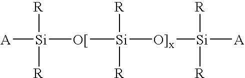
wherein each R is independently alkyl or aryl, x is an integer from about 7 to about 8,000, and each A represents a group which blocks the ends of the silicone chains.

8. The composition of claim 7 wherein each A is independently a methyl group, a methoxy group, an ethoxy group, a propoxy group, or an aryloxy group.

9. The composition of claim 7 wherein each R is independently a methyl group, an ethyl group, a propyl group, a phenyl group, a methylphenyl group, a phenylmethyl group, or a mixture thereof.

10. The composition of claim 1 wherein said silicone conditioning agent is polydimethyl siloxane, polydiethylsiloxane, polymethylphenylsiloxane, or a mixture thereof.

11. The composition of claim 1 wherein said cationic conditioning agent is a quaternary ammonium compound or an amino compound having at least one N-radical.

12. The composition of claim 11 wherein said cationic agent is ester containing quaternary ammonium compound.

13. The composition of claim 12 wherein said ester containing quaternary ammonium compound is a TEQ esterquat that comprises a mixture mono-(I), di-(II) and tri-ester (III) components of the following formulae: 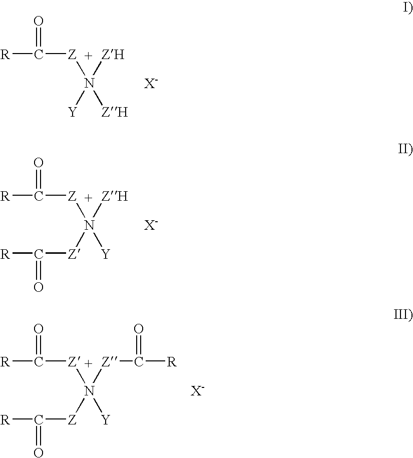
wherein:
Z, Z′ and Z″ are the same or different and are selected from straight or branched chain, optionally substituted oxyalkylene or polyoxyalkylene groups having from 2-6 carbon atoms, preferably 3-6 carbon atoms, where the oxyalkylene units number from about 1-10, preferably 1-5, and still more preferably 1-2; each R group is individually selected from straight or branched chain, saturated or unsaturated, optionally substituted alkyl groups having from 11 to 23 carbon atoms, Y is an alkylphenyl group or a straight or branched chain optionally substituted C₁ to C₆ alkyl or alkylene group; and X⁻ represents a softener compatible anion including but not limited to halogen, CH₃SO₄ or C₂H₅SO₄.

14. The composition of claim 12 wherein said ester containing quaternary ammonium compound is a DEQ esterquat of the formula 1 or 2:
[R′₄₋ₘ—N⁽⁺⁾—[(CH₂)ₙ—Y—R″]ₘX⁽⁻⁾ 1.
where each R′ is selected from the group consisting of C₁ to C₆ alkyl or hydroxyalkyl groups; each m is 2 or 3; each n is 1 to 4; each R″ is selected from the group consisting of C₁₂ to C₂₂ hydrocarbyl or substituted hydrocarbyl groups; each Y is —O—(O)C— or —C(O)—O and X⁻ is a compatable anion, or 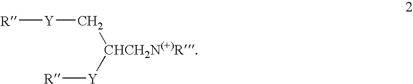
wherein each of Y, R′, R″ and X— have the meanings defined above and where R′″ is a hydrocarbon group containing 11 to 31 carbon atoms.

15. The composition of claim 12 wherein said ester containing quaternary ammonium compound is a diesterquat of formula 3: 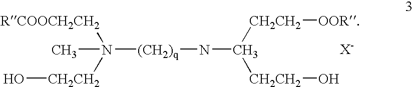
where each R″ is selected from the group consisting of C₁₂ to C₂₂ hydrocarbyl or substituted hydrocarbyl groups ; each Y is —O—(O)C— or —C(O)—O and X⁻ is a compatable anion and q is an integer of from 2-6.

16. The composition of claim 11 wherein said cationic conditioning agent is the salt of a primary, secondary, or tertiary fatty amine.

17. The composition of claim 16 wherein said amine is diethyl aminoethyl polyoxyethylene (5) laurate, coco-polyglyceryl-4 hydroxypropyl dihydroxy ethylamine, dihydroxyethyl tallowamine hydrochloride, or mixtures thereof.

18. The composition of claim 1 wherein said nonionic amphiphilic polymer containing at least one fatty chain and at least one hydrophilic unit is:
(1) hydroxypropyl guar modified with groups containing at least one fatty chain;
(2) polyether urethanes containing at least one fatty chain;
(3) copolymers of vinylpyrrolidone and hydrophobic monomers containing a fatty chain;
(4) copolymers of C₁-C₆ alkyl methacrylates, or acrylates and of amphiphilic monomers containing at least one fatty chain;
(5) copolymers of hydrophilic methacrylates or acrylates and of hydrophobic monomers containing at least one fatty chain,
or mixtures thereof.

19. The composition of claim 18 wherein said nonionic amphiphilic polymer containing at least one fatty chain and at least one hydrophilic unit is a polyether urethanes containing at least one fatty chain.

20. A hair conditioning composition that comprises:
(a) from about 0.5% to about 8%, by weight of at least one cationic conditioning agent;
(b) from about 0.05% to about 5%, by weight, of at least one dispersed, nonvolatile, insoluble, silicone conditioning agent having a viscosity, at 25° C., of from about 10 centipoise to about 100,000 centipoise;
(c) from about 0.2 to about 5% of at least one nonionic amphiphilic polymer containing at least one fatty chain and at least one hydrophilic unit, wherein said polymer is a polyether urethane;
(d) from about 4.5% to about 20% of at least one emulsifier,
(e) the remainder water.
